(19) United States
(12) Patent Application Publication
(10) Pub. No.: US 2012/0083708 A1
(43) Pub. Date: Apr. 5, 2012

(54) ADAPTIVE REAL-TIME SEIZURE PREDICTION SYSTEM AND METHOD

(76) Inventors: Pooja Rajdev, Bethesda, MD (US); Pedro Irazoqui, Lafayette, IN (US); Matthew Ward, Indianapolis, IN (US); Robert Worth, Indianapolis, IN (US)

(21) Appl. No.: 13/376,170

(22) PCT Filed: Jun. 2, 2010
(86) PCT No.: PCT/US10/37093

§ 371 Date: Dec. 2, 2011

Related U.S. Application Data
(60) Provisional application No. 61/183,408, filed on Jun. 2, 2009.

(51) Int. Cl. A61B 5/0476

(52) U.S. Cl. 600/544

(57) ABSTRACT

A real-time seizure prediction system. The system includes an implantable electrode configured to transmit an analog neuro-electrophysiological signal from a subject, an analog-to-digital converter configured to convert the analog neuro-electrophysiological signal to a digital neuro-electrophysiological signal based on a predetermined sampling rate, a processor configured to perform following steps during a period defined by the predetermined sampling rate: calculate a plurality of autocorrelation coefficients of the digital neuro-electrophysiological signal for a first predetermined number of samples, calculate a predicted future value of the digital neuro-electrophysiological data based on the plurality of autocorrelation coefficients and the first predetermined number of samples of the digital neuro-electrophysiological data, compare the predicted future value with an actual future value of the digital neuro-electrophysiological data to determine a prediction error, calculate a threshold based on a mean squared value of the prediction error for the first predetermined number of samples and based on a proportionality constant, generate a seizure prediction signal if the prediction error remains above the threshold for a second predetermined number of samples, and a warning device configured to receive the seizure prediction signal and generate an alert.

### CROSS-REFERENCE TO RELATED APPLICATIONS

This application claims priority from U.S. Provisional Patent Application No. 61/183,408, filed Jun. 2, 2009, the contents of which are incorporated herein by reference in their entirety.

### TECHNICAL FIELD

The present invention generally relates to seizure detection systems and methods and more particularly to seizure detection systems and methods capable of real-time seizure prediction.

### BACKGROUND

The need for reliable, automated seizure prediction is emphasized by 50 million people in the world suffering from epilepsy. Of those, 30% have not been able to gain any control over seizures resulting from epilepsy using current pharmacological treatment solutions. An epileptic seizure commonly manifests into physical attributes such as convulsion. However, prior to a clinical onset of the seizure changes in a neurosignal of a monitored subject in the form of electric discharges may be observed. Such changes are categorized as the electrographic onset of the seizure. Recognizing these changes may be helpful in predicting the seizure before the seizure manifests physical signs in the subject, e.g., convulsion. By predicting an oncoming seizure sufficiently early in the electrographic onset, it is possible to provide a stimulus to the subject that can prevent the seizure from spreading or can at least reduce the impact of the seizure on the subject.

The electrographic onset of a seizure is evidenced by a sustained number of low amplitude and high frequency electrical bursts in the neuro signal. Although not always readily identifiable, these low amplitude and high frequency bursts are different than other non-sustained electrical activity in the neurosignal.

Typically, neuron firing patterns (i.e., low amplitude and high frequency electrical bursts) are analyzed by obtaining average field potentials at the scalp of a subject by using an electroencephalogram (EEG). In addition, neuron firing patterns can be collected by obtaining local field potentials (LFP). The LFPs are obtained by strategically placing electrodes within brain matter, e.g., at the hippocampus.

In seizures with focal onset, the electric discharges tend to develop in the epileptogenic zone (most commonly the hippocampus in temporal lobe epilepsy, TLE) and spread to the cerebral cortex. Researchers in the prior art have shown that even if changes in the firing patterns of some neurons occur in the pre-seizure period, they are not expressed in the overall averaged field potentials observed in the EEG data. As a result, neuron firing patterns are only observed in an EEG after a seizure has spread to the cerebral cortex. The spread of the seizure to the cerebral cortex represents two shortcomings of using EEG as a predictor of seizure activity. First, the EEG data is inevitably distorted by the filtering and attenuation produced by the intervening layers of cerebrospinal fluid, tissue, skull, and scalp. These distortions make correlation of the EEG data with the neurophysiology of the focal point of the seizures difficult. Second, once the seizure has spread to the cerebral cortex, it is generally too late to provide a stimulus to stop the seizure.

Typically, prior art systems that extract information from EEG recordings in order to predict seizures were obtained from studies retrospectively (i.e., after the data was recorded). The computational complexity of algorithms associated with these systems, and training required by these algorithms represent at least two of the difficulties associated with implementing these algorithms in a real-time fashion. Supervised training refers to the technique requiring the patient to have at least one untreated seizure that is captured and analyzed by the system. As a result, an efficient, real-time, seizure prediction system has not yet been successfully developed.

Results from prior art systems is summarized in FIG. 9, entitled as “prior art”. Iasemidis et al. were the first to develop an automated algorithm that provided warning of impending seizures prospectively from multi-channel real-time EEG data. See Iasemidis, et al., Long-term prospective on-line real-time seizure prediction, Clin. Neurophysiol. 116 (3) (2005) 532-544. L. D. Iasemidis, et al., Adaptive epileptic seizure prediction system, IEEE Trans. Biomed. Eng. 50 (5) (2003) 616-627. L. D. L. D. Iasemidis, et al., Dynamical resetting of the human brain at epileptic seizures: application of nonlinear dynamics and global optimization techniques, IEEE Trans. Biomed. Eng. 51 (3) (2004) 493-506. L. D. Iasemidis, et al., Quadratic binary programming and dynamical system approach to determine the predictability of epileptic seizures, Journal of Combinatorial Optimization. Combin. Optim. 5 (1) (2001) 9-26. Using their previous results on dynamical entrainment of neurons in the epileptogenic focus (progressively converging Lypanov exponent), they proposed non-adaptive prospective algorithms that required reselecting the critical sites in the brain. See Dynamical resetting of the human brain at epileptic seizures: application of nonlinear dynamics and global optimization techniques. IEEE Trans Biomed Eng, 2004. 51(3): p. 493-506. They tested their first adaptive seizure prediction algorithm on five patients with refractory TLE and achieved a sensitivity of 84%, with a false positive rate of 0.12 per hour. Sensitivity is defined as the ratio of the total number of seizures predicted (or detected early) to the total number of seizures that actually occurred.

In 2005, they developed another algorithm that could automatically select the ‘best’ electrode channels using a zero/one optimization technique. See Long-term prospective on-line real-time seizure prediction. Clin Neurophysiol, 2005. 116(3): p. 532-44. In a study with two patients, they were able to predict the next seizure approximately 90 minutes prior to its onset with a success rate of 91.3%. However, this algorithm requires a seizure detection algorithm to run in parallel with the prediction algorithm and is contingent upon the onset time of the previous seizure since the update of the selected brain sites that are monitored for prediction of the next seizure occur at each seizure.

In 2005, D'Allesandro et al. tuned a real-time probabilistic neural network using the best combination of electrode sites and quantitative features for each patient. See M. D'Alessandro, et al., A multi-feature and multi-channel univariate selection process for seizure prediction, Clin. Neurophysiol. 116 (3) (2005) 506-516. Their method when tested provided a sensitivity of 100% on one patient, but failed on another patient. Additionally, their method also required training of the network by the leading (first) seizure in each patient. FIG. 9 provides a comparison of the sensitivities of some previously implemented algorithms. Although researchers using some of these algorithms have achieved high sensitivities, they have not been implemented and tested in real-time.

The field of seizure prediction holds great promise for patients who have not been able to gain control over their seizure. An effective system may include a closed-loop prosthesis that can intervene before the clinical onset of a seizure in order to stop progression of the seizure before it manifests its physical attributes. However, the system requires detection of an oncoming seizure with sufficient amount of time prior to the clinical onset in order to provide a stimulus to stop the seizure from spreading.

Therefore there is a need for an adaptive real-time system using local field potentials obtained from strategically implanted electrodes to predict onset of a seizure prior to manifestation of the seizure into the associated physical attributes.

### SUMMARY

A system and a method for real-time prediction of seizures have been developed.

In one form thereof, a real-time seizure prediction system includes an implantable electrode configured to transmit an analog neuro-electrophysiological signal from a subject, an analog-to-digital converter configured to convert the analog neuro-electrophysiological signal to a digital neuro-electrophysiological signal based on a predetermined sampling rate, and a processor. The processor is configured to perform following steps during a period defined by the predetermined sampling rate: calculate a plurality of autocorrelation coefficients of the digital neuro-electrophysiological signal for a first predetermined number of samples, calculate a predicted future value of the digital neuro-electrophysiological data based on the plurality of autocorrelation coefficients and the first predetermined number of samples of the digital neuro-electrophysiological data, compare the predicted future value with an actual future value of the digital neuro-electrophysiological data to determine a prediction error, calculate a threshold based on a mean squared value of the prediction error for the first predetermined number of samples and based on a proportionality constant, generate a seizure prediction signal if the prediction error remains above the threshold for a second predetermined number of samples, and a warning device configured to receive the seizure prediction signal and generate an alert.

In another form thereof, a method for predicting a seizure in real-time includes receiving an analog neuro-electrophysiological signal from an implantable electrode. The method also includes converting the analog neuro-electrophysiological signal to a digital neuro-electrophysiological signal based on a predetermined sampling rate, and calculating a plurality of autocorrelation coefficients of the digital neuro-electrophysiological signal for a first predetermined number of samples. The method further includes calculating a predicted future value of the digital neuro-electrophysiological data based on the plurality of autocorrelation coefficients and the first predetermined number of samples of the digital neuro-electrophysiological data, comparing the predicted future value with an actual future value of the digital neuro-electrophysiological data to determine a prediction error, calculating a threshold based on a mean squared value of the prediction error for the first predetermined number of samples and based on a proportionality constant, and generating a seizure prediction signal if the prediction error remains above the threshold for a second predetermined number of samples.

### BRIEF DESCRIPTION OF DRAWINGS

FIG. 1 is a block diagram of an exemplary system for data collection and processing of neurosignals including interface with external computing components;

FIG. 2 is a block diagram of a sub-system of the system of FIG. 1, provided for real-time implementation of a seizure prediction algorithm;

FIG. 3 is a diagram depicting local field potentials from a seizing rat;

FIG. 4 includes plots depicting (a) raw data, (b) mean, (c) variance, and (d) autocorrelation function of baseline data;

FIG. 5 is a diagram depicting relationship between prediction error and a prediction order for four rats;

FIG. 6 is a set of diagrams depicting (a) sensitivity vs. a proportionality constant; and (b) latency of prediction prior to clinical onset of seizure vs. the proportionality constant for each of the four rats;

FIG. 7 is a set of diagrams depicting raw and predicted data vs. time at different scales;

FIG. 8 is set of diagrams depicting raw neuro-signal vs. time, prediction error vs. time, prediction error envelope vs. time, and a binary decision (i.e., seizure/no-seizure) vs. time;

FIG. 9 is a diagram depicting sensitivities associated with various algorithms found in the prior art.

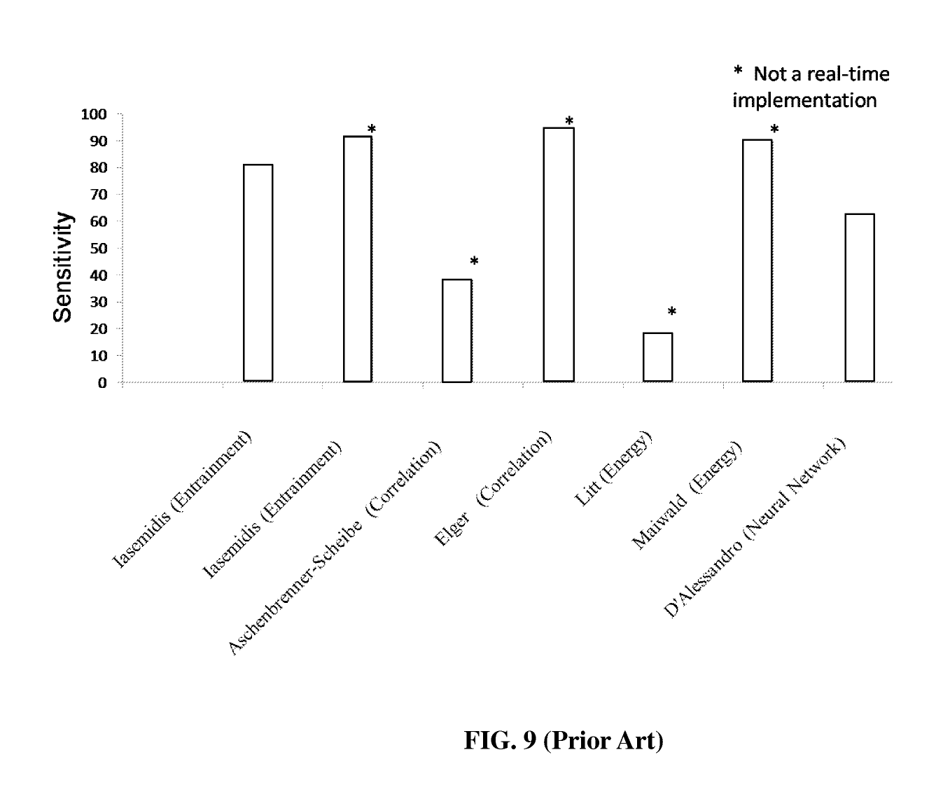

### DETAILED DESCRIPTION

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one of ordinary skill in the art to which this invention pertains.

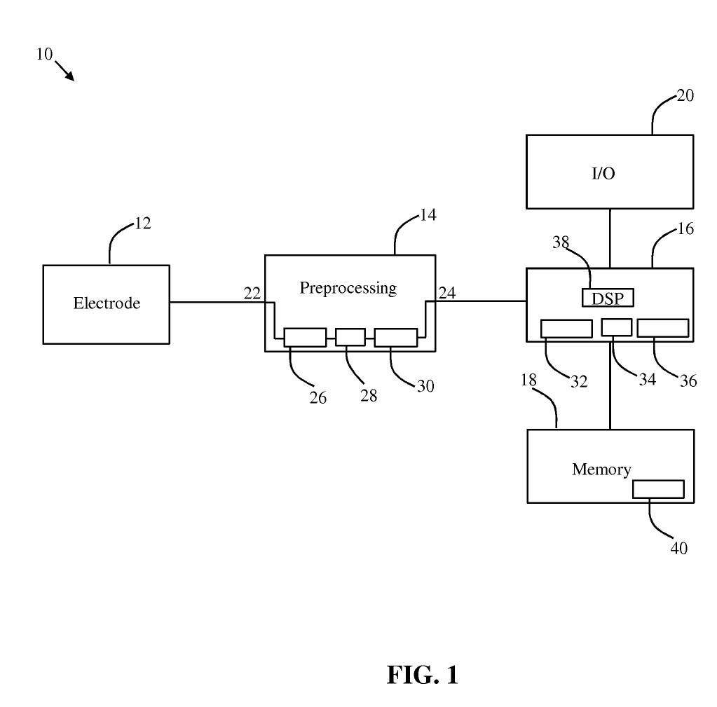

FIG. 1 depicts a representation of an exemplary seizure prediction (SP) system 10. The SP system 10 includes an electrode 12, a preprocessing circuit 14, a processing circuit 16, a memory circuit 18 and an input/output (I/O) device 20. The electrode 12 is coupled to the preprocessing circuit 14. The preprocessing circuit 14 is coupled to the processing circuit 16 which is coupled to the memory circuit 18 and the I/O device 20.

The preprocessing circuit 14 has an input 22 and an output 24 and is configured to process analog signals from the electrodes into digital signals suitable for processing by the processing circuit 16. Accordingly, the input 22 is coupled to the electrode 12. In this embodiment, the preprocessing circuit 14 further includes an amplifier 26, an infinite impulse response (IIR) bandpass filter 28, and an analog-to-digital converter (ADC) 30. The amplifier 26 is coupled to the input 22 on an input side of the amplifier 26 and coupled to the IIR bandpass filter 28 on an output side. The IIR bandpass filter 28 is coupled to the ADC 30 which is coupled to the output 24 of the preprocessing circuit 14. The ADC 30 may suitably be a digital signal processor (DSP) integrated circuit (IC). An example of the DSP IC is TMS320C6713DSK manufactured by Texas Instruments.

The processing circuit 16 is a processing circuit suitable for numerical calculations such as a DSP kit including a DSP IC 38 and its associated circuitry. An example of a DSP kit for the processing circuit 20 is C6713DSK provided by Texas Instruments which includes TMS320C6713T. The DSP IC 38 also manufactured by Texas Instruments. The processing circuit 20 further includes memory blocks 32 and 34, and an emulator interface block 36. The memory block 32 may be a volatile memory, e.g., synchronous dynamic random access memory (SDRAM), while memory block 34 may be a non-volatile memory, e.g., flash memory. The emulator interface block 36 couples the processing circuit 29 to an emulator for downloading instructions and other communications as well as for programming the memory block 34. The DSP IC 38 is coupled to the memory blocks 32 and 34 with address and data buses for transferring data into and out of the memory blocks. Similarly, the processing circuit 20 is in communication with the preprocessing circuit 14 by address and data buses to transfer data and commands between the two circuits 14 and 16. It will be appreciated that the physical details of the preprocessing circuit 14 and the processing circuit 16 can take other known forms.

The data communicated between the processing circuit 20 and the preprocessing circuit 14 includes configuration data, digital neuro-signals provided by the ADC 30, as well as other data. The preprocessing circuit 14 stores the configuration data that is communicated between the processing circuit 16 and the preprocessing circuit 14 in a memory block (not shown). The memory block (not shown) may include random access memory (RAM), read only memory (ROM), programmable read only memory (PROM), erasable programmable read only memory (EPROM), or electrically erasable read only memory (EEPROM), and other types of memory known in the art suitable for storing data. The data may be of the type that continuously changes, or of the type that changes during programming of the preprocessing circuit 14.

The memory circuit 18 may suitable be various memory and data storage elements associated with general purpose computing. Within the memory circuit 18 are various instructions in a program instruction block 40. The processing circuit 20 may be configured to execute the program instructions in block 40, or it may be configured to use program instructions stored in the non-volatile memory block 34, in order to carry out the various operations described fully below, as well as other operations.

The I/O device 20 may include a user interface, graphical user interface, keyboards, pointing devices, remote and/or local communication links, displays, and other devices that allow externally generated information to be provided to the SP system 10, and that allow internal information of the SP system 10 to be communicated externally. The I/O device 24 may also be configured to transfer user data to the processing circuit 16.

The electrode 12 may suitably be a single electrode or an electrode array that is/are connectable to animal tissue to detect neurosignals. The electrode 12 may be an implantable type or of a type that is adhereable to a skull.

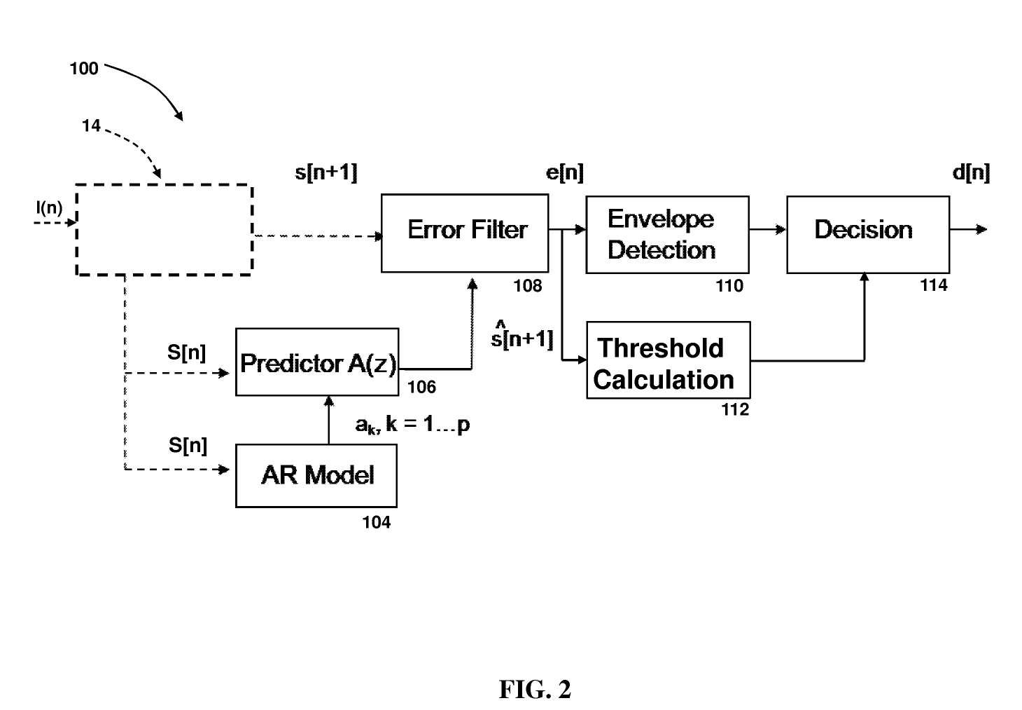

In FIG. 2, a block representation of steps performed in an algorithm 100 executed by the DSP IC 38 are depicted. The algorithm 100 includes an autoregressive (AR) model block (104), a predictor block (106), an error filter block (108), an error envelope detection block (110), a threshold calculation block (112), and a binary decision block (114). Also depicted in FIG. 2 is the preprocessing circuit 14 (shown in phantom). The preprocessing circuit 14 receives an analog raw neurosignal I(n) and provides an amplified bandpass filtered, and digitally converted digital samples S[n] to the blocks of the algorithm 100 (see also FIG. 1).

The preprocessing circuit 14 is operably configured to provide digitally sampled neurosignals S[n] to the AR model block (104) and the predictor block (106). The AR model block (104) is also coupled to the predictor block (106). The predictor block (106) is coupled to the error filter block (108). The error filter block (108) is operably coupled to the preprocessing circuit 14 to receive samples S[n+1] from the preprocessing circuit 14. The error filter block (108) is also coupled to the envelope detection block (110) as well as the threshold calculation block (110). The envelope detection block (110) and the threshold calculation block (112) both communicate with the binary decision block (114). The binary decision block (114) provides an output of the algorithm 100.

In operation, clinicians interface with the processing circuit 16 via the I/O device 20 in order to provide parameters that the processing circuit 16 uses internally and also parameters which are used to communicate with the preprocessing circuit 14. In addition, the I/O device 20 can also display data that is manipulated by the processing circuit 16. The processing circuit 16 receives the parameters from the I/O device 20 and communicates these parameters to the preprocessing circuit 14. The processing circuit 16 also communicates data from the preprocessing circuit 14 to the I/O device 24. These transfers of data take place in accordance with the program instructions that are stored in the memory block 18 or in the non-volatile memory block 34.

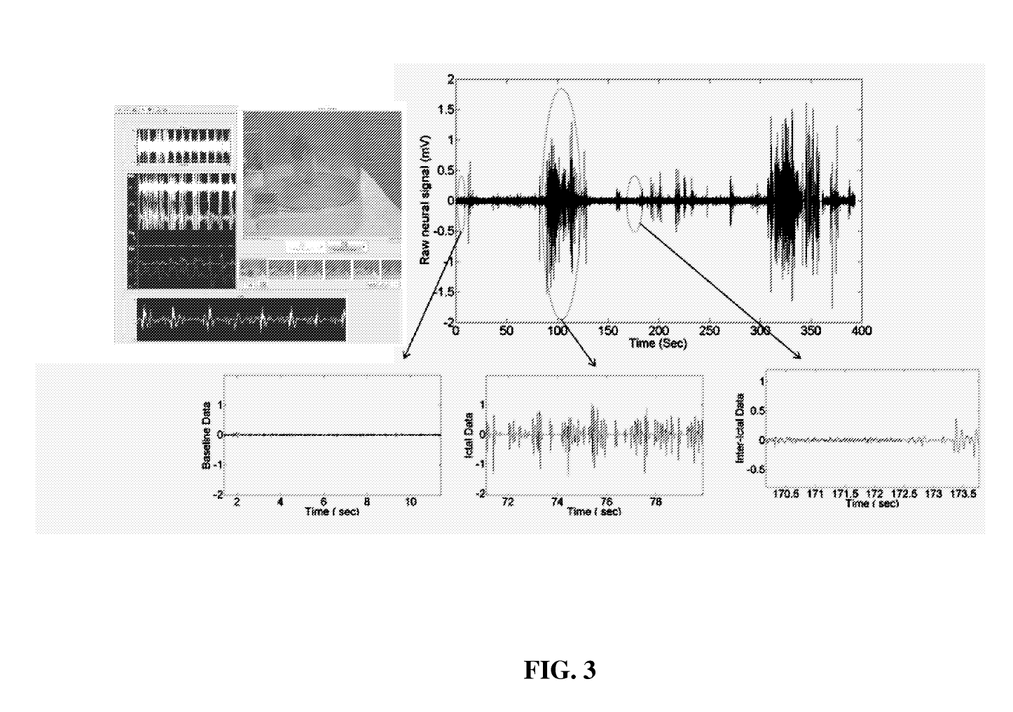

FIG. 3 depicts the experimental setup, showing a rat in a Plexiglas enclosure. Four Long Evans rats (250-350 gm) were anesthetized using 5% Isoflorane in 2 L/min O₂. Bipolar stainless steel Plastics1 electrodes were sterotaxically implanted into the dorsal dentate gyrus at the coordinates 4.0 mm posterior to bregma, 2.5 mm lateral to midline, and 3.3 mm ventral to dura. Prior to implanting the electrode, the clinician drilled a small hole in the skull of each rat at the required coordinates and placed 3 anchor screws around the hole. The clinician then wrapped a reference wire around the anchor screws. The electrodes were secured to the skull and the support screws using dental acrylate. After a 15-day post-surgical recovery period, the unrestrained and awake animals were injected intraperitoneally with doses of kainate to induce status epilepticus. A kainic acid solution (2.5 mg/ml in 0.9% NaCl) was administered in repeated, low doses (0.2 ml per 100 g) every hour until each rat experienced convulsive status epilepticus for over three hours. Seizure activity was carefully monitored throughout the procedure. Over the course of the treatment, the rat progressed from Class Ito Class V seizures (as classified by the modified Racine scale, known in the art).

Also depicted in FIG. 3 are raw neurosignals obtained from an implanted electrode in a seizing rat. Included in FIG. 3 are the raw neurosignals at different magnifications.

The preprocessing circuit 14 receives analog neurosignals from the electrode 12. Based on the parameters provided by the processing circuits 16, or alternatively based on fixed parameters within the preprocessing circuit 14, the preprocessing circuit 14 first amplifies the neurosignal by the amplifier 26, then filters the amplified analog neurosignals by the IIR bandpass filter 28 between 10 and 500 Hz, and then converts to a digital neurosignal by the ADC 30. In one exemplary embodiment, the amplifier 26 amplifies the raw neurosignal input by 100 times. The IIR bandpass filter 28 attenuates the low frequency noise and the high frequency artifacts that could lead to false positive detection of seizures. In one exemplary embodiment, the sampling rate of the ADC is set to 8 KHz.

The output of the preprocessing circuit 14, S[n], is provided to the AR model block (104) to calculate autocorrelation coefficients of the digital samples S[n]. An autocorrelation function is the cross-correlation of a signal with itself. The autocorrelation function is a mathematical function typically used for determining repeating patterns, e.g., periodic signals within what is otherwise noise. Typically, normalizing the autocorrelation function with mean and variance quantities generates autocorrelation coefficients. Using equation (1), below, the autocorrelation coefficients can be determined.

$${\sum }_{l = 1}^{p} \hat{a} [ k ] {\hat{R}}_{ss} [ k - l ] = - {\hat{R}}_{ss} [ k ] , k = 1 , 2 , … , p , \qquad ( 1 )$$

wherein â[k] are the autocorrelation coefficients,
p is a the order of the autocorrelation function,
k is an index for the order of the autocorrelation function, and
\hat{R}ₛₛ is an estimate of the autocorrelation function obtained by:

$${\hat{R}}_{ss} [ k ] = \frac{1}{N} {\sum }_{n = 0}^{N - 1 - \vert k \vert} s [ n ] s [ n + \vert k \vert ] , \vert k \vert \leq N - 1 , \qquad ( 2 )$$

wherein N is the number of data points in the digital samples S[n].

The autocorrelation coefficients are continuously calculated based on the incoming digital samples S[n].

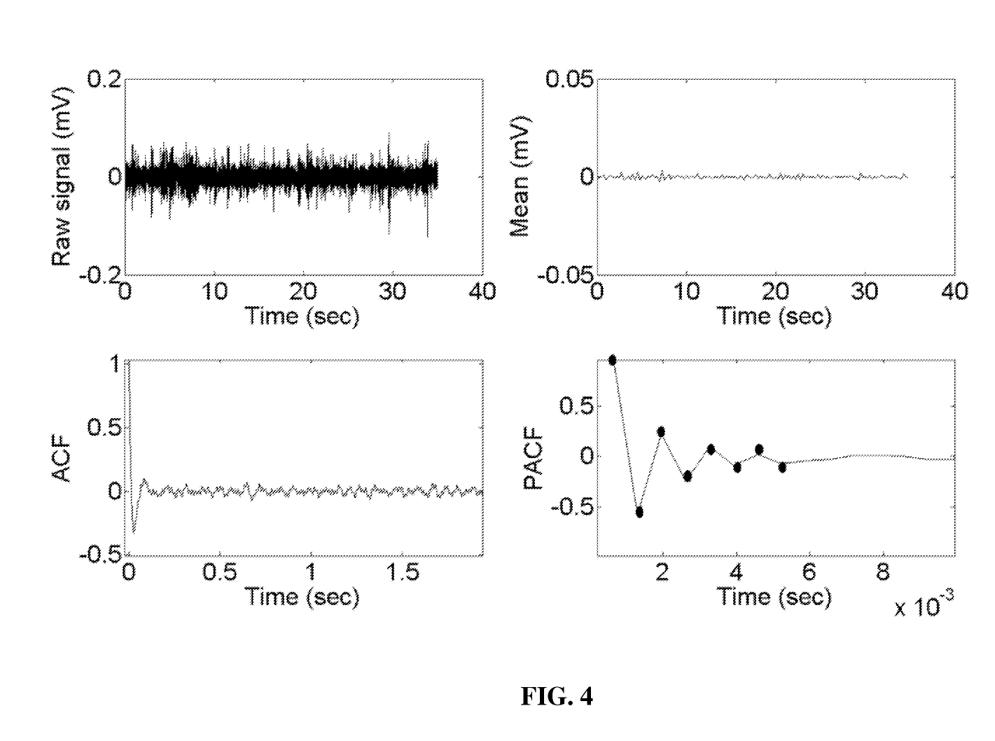

Stationary time series with short-term correlation provide autocorrelation coefficients that have few large value coefficients followed by exponentially decaying coefficients that approach zero. As a result, a time series can be modeled as an AR process of order p if the autocorrelation function (ACF) decays exponentially, while the partial autocorrelation function (PACF) cuts off after p lags, where a lag denotes a sample shift. FIG. 4 includes diagrams of raw neurosignal time series vs. time, mean value of the time series vs. time, the ACF of the time series vs. time, and PACF vs. time. FIG. 4 shows that the time series can be considered quasi-stationary since the mean of the signal is almost constant over time, while the ACF plot decreases exponentially to approach zero. The values of the PACF are significantly non-zero only for a few shifts (about 8), thereby, encouraging an AR model of the time series of order 8.

The AR model block (104) provides the autocorrelation coefficients (i.e., from equation (1)) to the predictor block (106). The predictor block (106) receives the autocorrelation coefficients and the digital samples S[n] to predict the future valve of the time series (i.e., Ŝ(n+1)). The predictor block (106) uses equation (3), below, to predict the value of the Ŝ(n+1):

$$\hat{s} [ n + 1 ] = {\sum }_{k = 0}^{p} a [ k ] s [ n - k ] , \qquad ( 3 )$$

wherein a[k] are the autocorrelation coefficients,
p is the order of the autocorrelation function,
and S[n−k] are past values of the digital samples S[n].

Determining the order (p) of the prediction model of the predictor block (106), requires careful consideration. The order of operations in the predictor block (106) is based on a squared value of p (i.e., p²). Therefore, the order cannot be exceedingly large in order to maintain a feasible real-time operation (i.e., being able to calculate the next predicted value of the time series, Ŝ(n+1), during one period of the sampling rate of the ADC 30, see FIG. 1).

The Akaike information criterion (AIC) provides one method to determine the model order p. The order p can be determined by minimizing an information theoretic function of p as provided in equation (4), below:

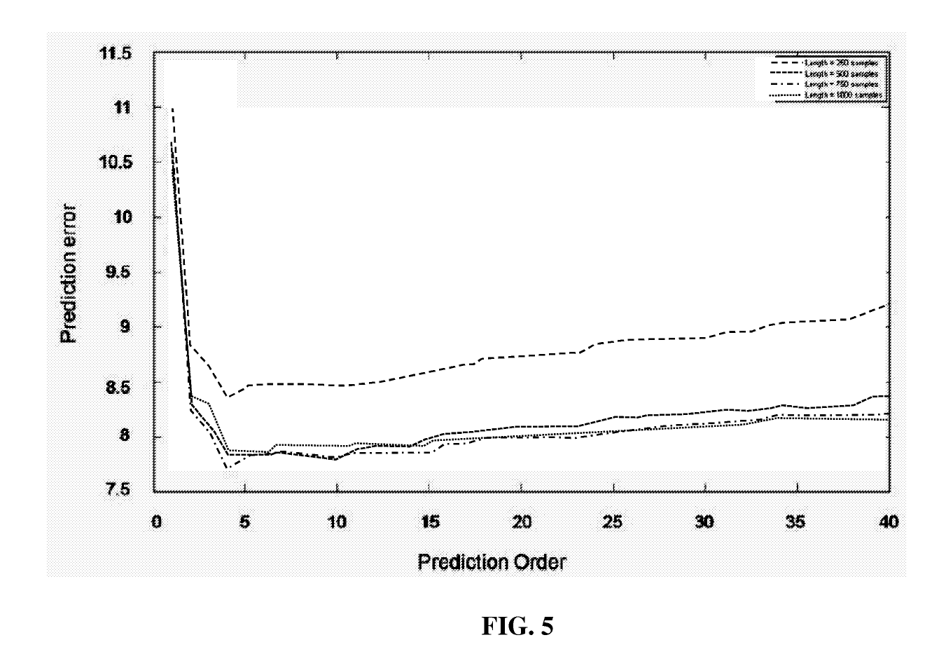

AIC(p)=lnσ²+2p/N (4)

wherein σ is variance between the predicted values and actual values of the time series. As the model order p is increased, the variance term σ² decreases while the term 2p/N increases. Minimizing this function provides a model order of 10 for a prediction length of 500 samples for the predictor block 106. FIG. 5 is a diagram depicting minimization of the equation (4), expressed as prediction error vs. prediction order.

Once the future value of the digital samples S[n] is predicted (i.e., Ŝ(n+1)), the value is provided to the error filter block (108) in order to determine the error in the prediction (i.e., e(n)).

The error filter block calculates a squared function of the error based on equation (5), below:

$$\begin{matrix}e ( n ) = ɛ [ n + 1 ] \\ = {( s [ n + 1 ] - \hat{s} [ n + 1 ] )}^{2} \\ = {( s [ n + 1 ] - {\sum }_{k = 0}^{p} a [ k ] s [ n - k ] )}^{2} ,\end{matrix} \qquad ( 5 )$$

wherein S[n+1] is the actual future value of the time series, and
Ŝ(n+1) is the predicted future value.

Autoregressive prediction methods can only effectively model stationary signals. The digital samples S[n] include quasi-stationary baseline signal and superimposed transient non-stationarities. Therefore, the prediction algorithm performs better on the baseline signals. Prior to the clinical onset of a seizure (i.e., the portion of the seizure accompanied by physical attributes, e.g., convulsion), the time series rapidly changes. As a result, the autoregressive process does not accurately predict system behavior, yielding continuously increasing prediction error values.

The error filter block (108) provides the error function (i.e., e(n)) to the envelope detection block (110) and threshold calculation block (112). The envelope detection block (110) extracts the envelope of the error function by a Hilbert transform, known in the art.

The threshold calculation block (112) calculates a threshold that is provided to and used by the binary decision block (114). The threshold is calculated based on equation (6), below:

$$T = λ \frac{1}{N} {\sum }_{n = 1}^{N} {e}^{2} [ n ] , \qquad ( 6 )$$

wherein where N is the number of samples in the window length,
λ is a scaling factor, and
e²[n] is the squared prediction error signal.

The binary decision block (114) receives both the extracted envelope information of the error signal and the threshold. A seizure is predicted when the prediction error envelope exceeds the threshold for k successive intervals, where k is a pre-determined number of samples. In addition to the error envelope exceeding the threshold for the predetermined number of samples, concurrently the amplitude of the digital samples S[n] must be below a predetermined threshold T₁. The threshold T₁ helps to avoid predicting a false seizure when there are artifacts, e.g., due to motion, present in the digital samples S[n] which cause an increase in the error signal e(n), particularly, due to high amplitudes of these artifacts. In other words, the binary decision block (114) is mainly searching for an increase in the error signal e(n) with the time series being below an amplitude threshold (T₁). In equation (5), above, λ is a proportionality constant that can affect the success of predicting a seizure within the binary decision block (114).

A seizure prediction is considered to be successful if the seizure is predicted before its electrographic onset (defined by spike and wave complexes, increases in LFP amplitude and confirmed by video recordings), or detected in its early stages (60 seconds). The Seizure Warning Horizon (SWH) is defined as the time window following the warning during which and event will occur and which is typically set to 3 minutes.

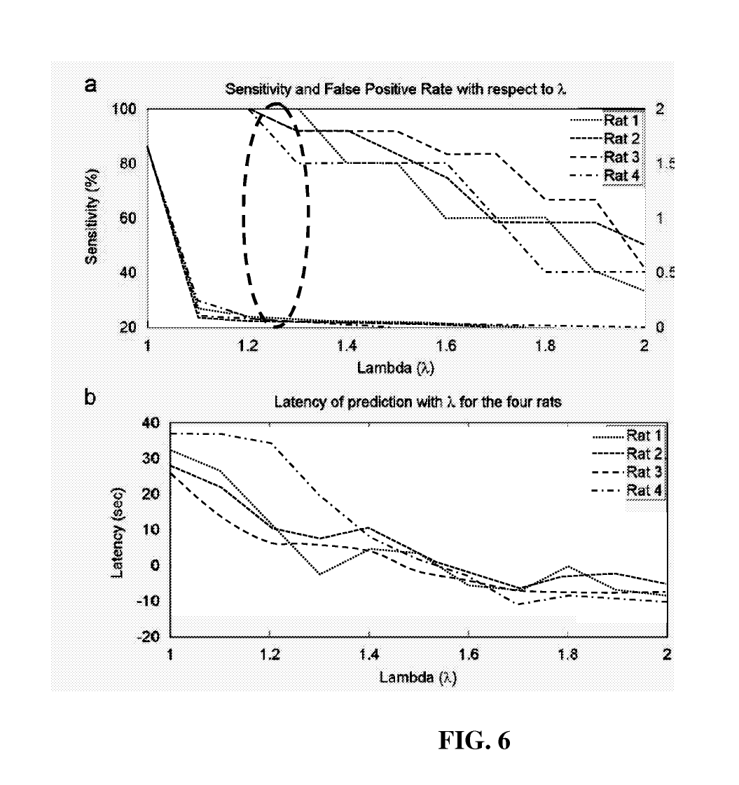

The sensitivity of the algorithm is defined as the fraction of the total number of seizures predicted (or detected early) to the total number of seizures that actually occurred. The false positive rate (FPR) is defined as the average number of false positives per hour. Latency is defined as the time difference (in sec) between a seizure warning and the electrographic onset of the seizure. To increase sensitivity, certain parameters of the seizure prediction algorithm 100 may be adjusted. However, some adjustments may also influences the FPR and latency. Effects of adjusting one of these parameters, λ (i.e., the proportionality constant), on the sensitivity and latency is depicted in FIG. 6. Output parameters such as sensitivity, FPR and latency all decreased as λ was increased. The trend was consistent among all four rats. For the values of λ between 1.1 and 1.3, 80-100% sensitivity was observed with an FPR of less than 0.02/min. Therefore, the choice of λ between 1.1 and 1.3 provides robust results for the output parameters.

### Implementations

The performance of this algorithm was evaluated using both Matlab evaluation software on a personal computer and the TMS320C6713 DSP used for the DSP IC 38.

The Matlab analysis showed that the prediction error build-up started seconds before the electrographic onset of the seizure. The prediction error gradually increased prior to a seizure. This gradual increase reflects a change in the underlying AR model. Also, the prediction error values drastically increase with the amplitude of the signal. This increase in signal amplitude is partly due to synchronization of a large number of firing neurons and is partly an artifact caused by the movement of the rats.

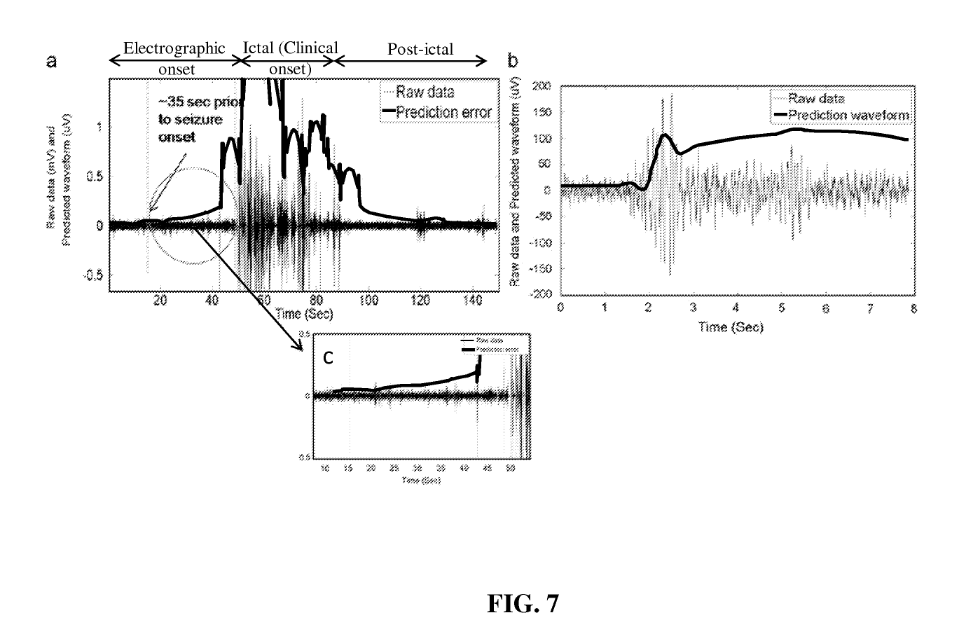

FIG. 7 includes diagram showing raw neurosignals vs. time as well as prediction error vs. time. FIG. 7 depict characteristic examples of differences between an artifact (noise peak or motion artifact) and an increase in amplitude that occurs at the onset of the seizure. The reason for this difference is that only seizures displayed a steady accumulation of prediction error, whereas noise or motion artifact peaks were an abrupt increase in prediction error, as well as amplitude. For example in the diagram titled “b”, between 2 and 3 seconds a sudden increase in the prediction error followed by a sudden decrease is observed. The sudden increase-decrease pattern may be due to a motion artifact occurring at about 2.5 seconds. However, at about 10 seconds, a continuous increase in the prediction error is observed, as predicted in the diagram entitled “c”. Therefore, the increase in the prediction error begins approximately 35 sec prior to the clinical onset of the seizure.

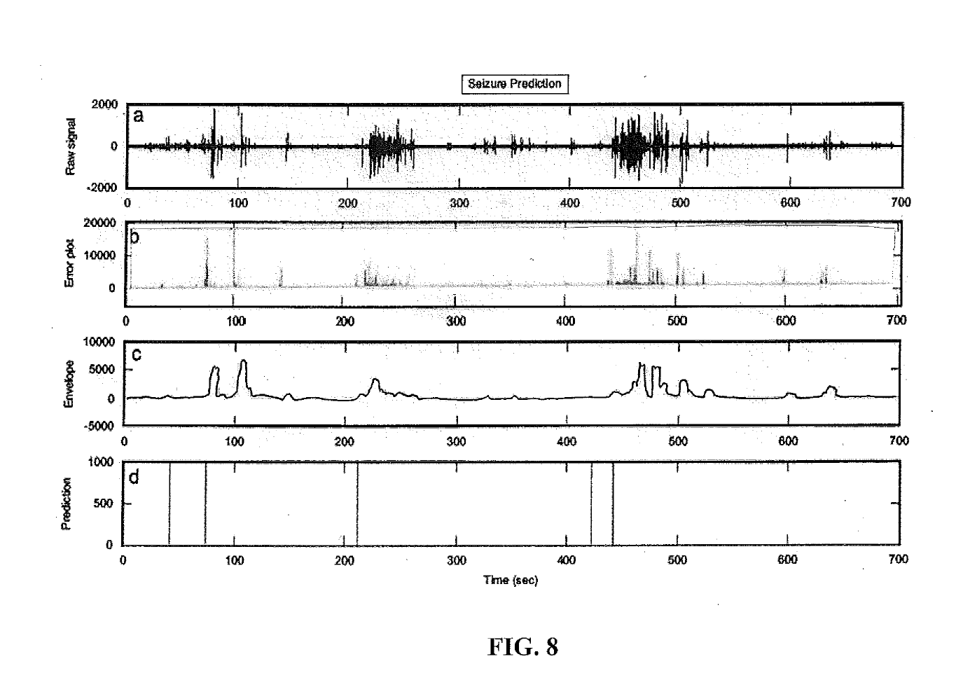

FIG. 8 includes diagrams showing raw neurosignals vs. time over a long period of time, prediction error over the same period, prediction envelope over the same period, and the binary decision over the same period. The squared of the prediction error (i.e., the envelope) in the baseline, pre-ictal and ictal regions of each seizure have significantly different values. These different regions can be demarcated using known threshold algorithms. In one embodiment, an adaptive threshold that is proportional to the mean squared energy of the prediction envelope can be used. A seizure is predicted when the error exceeds the thresholds for k successive intervals (k can be set to 8, in one embodiment). The requirement for k successive intervals condition ensures that short duration artifacts are not flagged as seizures.

Results from the Matlab investigation are tabulated and provided in Table 1.

TABLE 1
Performance of the predictor using Matlab

| | | | | Selec- | Median | Mean | Std of |
|---|---|---|---|---|---|---|---|
| | # of | Sensitivity | | tivity | Latency | latency | latency |
| ID | Seizures | (%) | FP/Min | (%) | (sec) | (sec) | (sec) |
| 1 | 32 | 96.87 | 0.0064 | 96.87 | 19.96 | 26.02 | 20.84 |
| 2 | 27 | 96.29 | 0.0095 | 92.85 | 34.79 | 33.29 | 18.12 |
| 3 | 25 | 88 | 0.0063 | 95.65 | 29.185 | 31.34 | 19.61 |
| 4 | 25 | 96 | 0.0143 | 92.30 | 13.824 | 15.51 | 10.50 |

As shown in Table 2, testing of this algorithm on kainate treated rats resulted in prediction of seizures 27±17 seconds before clinical onset, with 94% sensitivity and a false positive rate of 0.009 per minute.

Real-time performance was evaluated by coding the algorithm in the C computer language and uploading an executable version of the program onto the DSP IC 38 (16713). Prediction length was kept to be about 2042 samples (at 8 kHz) and the model order was 10 (See Eq. 4). Results of the real-time experiments are provided in Table 2.

TABLE 2
Performance of the predictor using the DSP IC 38 (16713)

| | | | | | Median | Mean | Std of |
|---|---|---|---|---|---|---|---|
| | # of | Sensitivity | FP/ | Selectivity | Latency | latency | latency |
| ID | Seizures | (%) | Min | (%) | (sec) | (sec) | (sec) |
| 1 | 14 | 92.85 | 0.115 | 76.47 | 6.33 | 5.63 | 4.29 |
| 2 | 14 | 92.85 | 0.077 | 81.25 | 7.68 | 8.35 | 7.63 |
| 3 | 24 | 91.67 | 0.074 | 88 | 5.18 | 6.35 | 4.87 |
| 4 | 18 | 88.89 | 0.080 | 88.89 | 6.91 | 7.24 | 5.34 |

As shown in Table 2, real-time testing resulted in prediction of seizures 6.7±5.6 seconds before its clinical onset, with 92% sensitivity and a false positive rate of 0.08 per minute.

The current approach threshold-based algorithm provides a comparison of the mean energy of the prediction error signal in a present window to a scaled version of the mean energy of a previous data segments. This comparison allows the algorithm to check for build-ups and compare current values to previous baseline values rather than instantaneous thresholds. Trained professionals could change the value of λ (proportionality constant) to increase latency or sensitivity or decrease false positive rates. More sophisticated thresholding algorithms could also be used to make the binary decision.

In addition, the proposed algorithm is not excessively demanding from a computational measure and is implementable on a DSP to provide a real-time seizure prediction utility. Also, supervised training, as seen in the prior art, is not required as the adaptive nature of the algorithm recalculates the coefficients to continually update the coefficients.

Those skilled in the art will recognize that numerous modifications can be made to the specific implementations described above. Therefore, the following claims are not to be limited to the specific embodiments illustrated and described above. The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

1. A real-time seizure prediction system, comprising:
an implantable electrode configured to transmit an analog neuro-electrophysiological signal from a subject;
an analog-to-digital converter configured to convert the analog neuro-electrophysiological signal to a digital neuro-electrophysiological signal based on a predetermined sampling rate;
a processor configured to perform following steps during a period defined by the predetermined sampling rate:
calculate a plurality of autocorrelation coefficients of the digital neuro-electrophysiological signal for a first predetermined number of samples;
calculate a predicted future value of the digital neuro-electrophysiological data based on the plurality of autocorrelation coefficients and the first predetermined number of samples of the digital neuro-electrophysiological data;
compare the predicted future value with an actual future value of the digital neuro-electrophysiological data to determine a prediction error;
calculate a threshold based on a mean squared value of the prediction error for the first predetermined number of samples and based on a proportionality constant;
generate a seizure prediction signal if the prediction error remains above the threshold for a second predetermined number of samples;
and
a warning device configured to receive the seizure prediction signal and generate an alert.

2. The real-time seizure prediction system of claim 1, wherein the plurality of autocorrelation coefficients of the digital neuro-electrophysiological signal are based on a third predetermined number of samples.

3. The real-time seizure prediction system of claim 2, wherein the third predetermined number of samples is determined by minimizing an information function.

4. The real-time seizure prediction system of claim 3, the information function is defined by: lnσ²+2p/N, wherein σ is variance defined by the difference between the predicted future values and actual future values of the digital neuro-electrophysiological data, p is the third predetermined number of samples, and N is the first predetermined number of samples.

5. The real-time seizure prediction system of claim 4, wherein the third predetermined number of samples is 10 and the first predetermined number of samples is 500.

6. The real-time seizure prediction system of claim 5, the plurality of autocorrelation coefficients of the digital neuro-electrophysiological signal is calculated by
$${\sum }_{l = 1}^{p} \hat{a} [ k ] {\hat{R}}_{ss} [ k - l ] = - {\hat{R}}_{ss} [ k ] ,$$
wherein â[k] represent the plurality of autocorrelation coefficients,
$${\hat{R}}_{ss} [ k ] = \frac{1}{N} {\sum }_{n = 0}^{N - 1 - \vert K \vert} s [ n ] s [ n + \vert k \vert ] ,$$
k=1, 2, . . . p, and p represents the third predetermined number of samples.

7. The real-time seizure prediction system of claim 1, wherein the alert is generated a predetermined amount of time prior to onset of a physiological event.

8. A method for predicting a seizure in real-time, comprising:
receiving an analog neuro-electrophysiological signal from an implantable electrode;
converting the analog neuro-electrophysiological signal to a digital neuro-electrophysiological signal based on a predetermined sampling rate;
calculating a plurality of autocorrelation coefficients of the digital neuro-electrophysiological signal for a first predetermined number of samples;
calculating a predicted future value of the digital neuro-electrophysiological data based on the plurality of autocorrelation coefficients and the first predetermined number of samples of the digital neuro-electrophysiological data;
comparing the predicted future value with an actual future value of the digital neuro-electrophysiological data to determine a prediction error;
calculating a threshold based on a mean squared value of the prediction error for the first predetermined number of samples and based on a proportionality constant; and
generating a seizure prediction signal if the prediction error remains above the threshold for a second predetermined number of samples.

9. The method of claim 8, wherein the steps are performed within a period defined by the predetermined sampling rate.

10. The method of claim 9, wherein the plurality of autocorrelation coefficients of the digital neuro-electrophysiological signal are based on a third predetermined number of samples.

11. The method of claim 10, wherein the third predetermined number of samples is determined by minimizing an information function.

12. The method of claim 11, the information function is defined by: lnσ²+2p/N, wherein σ is variance defined by the difference between the predicted future values and actual future values of the digital neuro-electrophysiological data, p is the third predetermined number of samples, and N is the first predetermined number of samples.

13. The method of claim 12, wherein the third predetermined number of samples is 10 and the first predetermined number of samples is 500.

14. The method of claim 13, the plurality of autocorrelation coefficients of the digital neuro-electrophysiological signal is calculated by
$${\sum }_{l = 1}^{p} \hat{a} [ k ] {\hat{R}}_{ss} [ k - l ] = - {\hat{R}}_{ss} [ k ] ,$$
wherein â[k] represent the plurality of autocorrelation coefficients,
$${\hat{R}}_{ss} [ k ] = \frac{1}{N} {\sum }_{n = 0}^{N - 1 - \vert K \vert} s [ n ] s [ n + \vert k \vert ] ,$$
k=1, 2, . . . p, and p represents the third predetermined number of samples.

15. The method of claim 8, further comprising generating an alert corresponding to generation of the seizure prediction signal. The method of claim 15, wherein the alert is generated a predetermined amount of time prior to onset of a physiological event.
